(19) United States
(12) Patent Application Publication
(10) Pub. No.: US 2012/0010164 A1
(43) Pub. Date: Jan. 12, 2012

(54) ANTIVIRAL AGENTS

(76) Inventors: Vincenzo Surnma, Rome (IT); Maria Emilia Di Francesco, Brookline, MA (US); Marco Pompei, Brussels (BE)

(21) Appl. No.: 13/145,456

(22) PCT Filed: Jan. 19, 2010
(86) PCT No.: PCT/EP10/50578

§ 371 Date: Sep. 28, 2011

(30) Foreign Application Priority Data
Jan. 20, 2009 (GB) 0900914.3

(51) Int. Cl. A61K 31/7064; A61P 31/14; A61K 31/7076; C07H 19/14; C07H 19/19

(52) U.S. Cl. 514/45; 536/27.2; 536/27.22; 514/49

(57) ABSTRACT

Compounds of structural formula (I): and pharmaceutically acceptable salts thereof; as defined herein, are described for use in the prevention and/or treatment of HCV infections. Novel compounds of the formula (I) and pharmaceutical formulations containing them are also described.

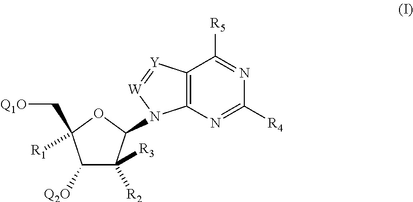

The present invention is concerned with nucleoside and nucleotide derivatives, their synthesis, and their use as inhibitors of RNA-dependent RNA viral polymerases. The compounds of the present invention are inhibitors of RNA-dependent RNA viral replication and are therefore useful for the treatment of RNA-dependent RNA viral infections. They are particularly useful as inhibitors of hepatitis C virus (HCV) NS5B polymerase, as inhibitors of HCV replication, and for the treatment of hepatitis C infection.

Hepatitis C virus (HCV) infection is a major health problem that leads to chronic liver disease, such as cirrhosis and hepatocellular carcinoma, in a substantial number of infected individuals, estimated to be 2-15% of the world's population. There are an estimated 4.5 million infected people in the United States alone, according to the U.S. Center for Disease Control. According to the World Health Organization, there are more than 200 million infected individuals worldwide, with at least 3 to 4 million people being infected each year. Once infected, about 20% of people clear the virus, but the rest harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their off-spring. Current treatments for HCV infection, which are restricted to immunotherapy with recombinant interferon-α alone or in combination with the nucleoside analog ribavirin, are of limited clinical benefit. Moreover, there is no established vaccine for HCV. Consequently, there is an urgent need for improved therapeutic agents that effectively combat chronic HCV infection. Different approaches to HCV therapy have been taken, which include the inhibition of viral serine proteinase (NS3 protease), helicase, and RNA-dependent RNA polymerase (NS5B), and the development of a vaccine.

The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. NS5B polymerase is therefore considered to be an essential component in the HCV replication complex [see K. Ishi, et al., “Expression of Hepatitis C Virus NS5B Protein: Characterization of Its RNA Polymerase Activity and RNA Binding,” Hepatology, 29: 1227-1235 (1999) and V. Lohmann, et al., “Biochemical and Kinetic Analyses of NS5B RNA-Dependent RNA Polymerase of the Hepatitis C Virus,” Virology, 249: 108-118 (1998)] Inhibition of HCV NS5B polymerase prevents formation of the double-stranded HCV RNA and therefore constitutes an attractive approach to the development of HCV-specific antiviral therapies.

The development of inhibitors of HCV NS5B polymerase with potential for the treatment of HCV infection has been reviewed in M. P. Walker et al., “Promising candidates for the treatment of chronic hepatitis C,” Expert Opin. Invest. Drugs, 12: 1269-1280 (2003) and in P. Hoffmann et al., “Recent patents on experimental therapy for hepatitis C virus infection (1999-2002), “Expert Opin. Ther. Patents,” 13: 1707-1723 (2003). The activity of purine ribonucleosides against HCV polymerase was reported by A. E. Eldrup et al., “Structure-Activity Relationship of Purine Ribonucleosides for Inhibition of HCV RNA-Dependent RNA Polymerase,” J. Med. Chem., 47: 2283-2295 (2004). There is a continuing need for structurally diverse nucleoside derivatives as inhibitors of HCV polymerase as therapeutic approaches for HCV therapy.

Chinese patent application CN 10117742 discloses nucleoside derivatives of the formula:

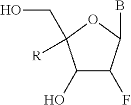

wherein R is methyl, nitrile, azido or ethynyl, and B is an optionally substituted pyrimidine, purine or 7-deazapurine base as having antiviral activity.

The compound wherein R is azido and b is uridine is specifically disclosed and reported to have activity against the HCV virus.

Journal of Biological Chemistry (208), 283(4), 2167-2175 discloses that c2′-dexy 4′-azido nucleoside analogues containing a substituted pyrimidine base are“highly potent inhibitors of HCV replication”.

The present invention provides a novel class of nucleosides and nucleotides that are potent inhibitors of RNA-dependent RNA viral replication and in particular HCV replication.

The present invention relates to a compound of structural formula (I):

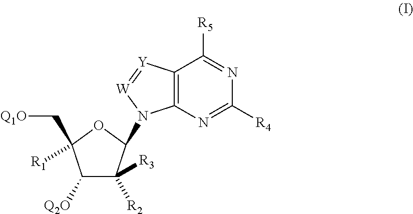

and pharmaceutically acceptable salts thereof; wherein: Y is a group CR⁶ wherein R⁶ is hydrogen, CHO, nitrile, ethynyl, a group CONH₂ optionally substituted by one or two C₁₋₃ aliphatic groups, or a C₁₋₃ aliphatic group optionally substituted by fluoro or R⁶ is amino optionally substituted by COR⁷, wherein R⁷ is a C₁₋₆ aliphatic group or phenyl or Y is linked to R⁵ to form a tricyclic ring:

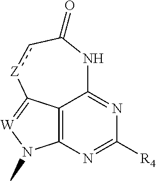

wherein the dotted line represents a single or double bond, Z represents CH when the dotted line represents a double bond or O or CH₂ when the dotted line represents a single bond;

W is N or CH;

R¹ is azido, ethynyl, nitrile or a C₁₋₃ aliphatic group optionally substituted by fluoro;

R² is hydrogen or fluoro;

R³ is hydrogen, fluoro or a hydroxy or C₁₋₃ alkoxy group or a C₁₋₃ aliphatic group optionally substituted by fluoro;

R⁴ is hydrogen, amino or hydroxyl;

R⁵ is hydroxyl or amino;

Q¹ is hydrogen or a mono-, di- or tri-phosphate group or a protecting group Q³; and

Q² is hydrogen or a protecting group Q⁴;

for use in the prevention and/or treatment of HCV infections.

Suitably R¹ is azido or ethynyl and most suitably azido.

Suitably R² is hydrogen.

Suitably R³ is fluoro.

Suitably R⁴ is hydrogen.

Suitably R⁵ is amino.

Suitably Y is CH.

Suitably W is CH.

Suitable groups Q³ and Q⁴ are well known to those skilled in the art, for example those described in WO2006/065335 and PCT/EP2008/056128 which are incorporated herein by reference. For example Q³ may be C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl or a monophosphate prodrug residue

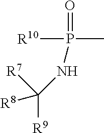

R⁷ is hydrogen, C₁-6alkyl optionally substituted with one substituent selected from the group consisting of fluoro, hydroxy, methoxy, amino, carboxy, carbamoyl, guanidino, mercapto, methylthio, 1H-imidazolyl, and 1H-indol-3-yl; or R⁷ is phenyl, benzyl or phenethyl each optionally substituted with one to two substituents independently selected from the group consisting of halogen, hydroxy, and methoxy;
R⁸ is hydrogen or methyl;
or R⁷ and R⁸ together with the carbon atom to which they attached form a 3- to 6-membered aliphatic spirocyclic ring system;
R⁹ is aryl, arylalkyl, heteroaryl or

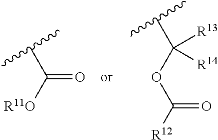

wherein R¹¹ is C₁₋₁₆alkyl, C₂₋₂₀alkenyl, (CH₂)₀₋₄C₇₋₉cycloalkyl, (CH₂)₀₋₄C₃₋₉cycloalkenyl or adamantly each optionally substituted with one to three substituents independently selected from halogen, hydroxy, carboxy, C₁₋₄alkoxy, trifluoromethyl and (CH₂)₀₋₄NR¹⁵R¹⁶ wherein R¹⁵ and R¹⁶ are independently selected from hydrogen and C₁₋₆alkyl; or R¹⁵ and R¹⁶, together with the nitrogen atom to which they are attached form a 4- to 7-membered heterocyclic ring optionally containing 1 or 2 more heteroatoms selected from N, O and S, which ring is optionally substituted by C₁₋₆ alkyl;
R¹⁰ is hydroxy or a group OR¹⁶ wherein R¹⁶ is CH₂OC(O)R¹⁷ or CH₂CH₂SR¹⁷ where R¹⁷ is C₁₋₆ alkylcarbonyl optionally substituted by a hydroxyl group or R¹⁶ is (CH₂)₂₋₄—O—(CH₂)₁₋₁₇CH₃, or an aromatic ring selected from phenyl, naphthyl, pyridinyl, pyrimidinyl, pyrazinyl, pyridazinyl, indolyl, quinolinyl, or isoquinolinyl, wherein the aromatic ring is optionally substituted with one to five substituents independently selected from the group consisting of halogen, C₁₋₄ alkyl, C₁₋₄ alkoxy, C₁-4 alkylthio, cyano, nitro, amino, carboxy, trifluoromethyl, trifluoromethoxy, C₁₋₄ alkylamino, di(C₁₋₄ alkyl)amino, C₁₋₄ alkylcarbonyl, C₁₋₄ alkylcarbonyloxy, and C₁₋₄ alkyloxycarbonyl; or R¹⁰ and Q⁴ form a bond to make a cyclic phosphate group; R¹² is C₆₋₁₆alkyl, C₂₋₂₀alkenyl, (CH₂)₀₋₂C₇₋₉cycloalkyl, (CH₂)₀₋₂C₃₋₉cycloalkenyl, OC₁₋₆alkyl or adamantyl; and
R¹³ and R¹⁴ are independently selected from hydrogen and C₁₋₆alkyl;
or R¹³ and R¹⁴ together with the carbon atom to which they attached form a 3- to 6-membered aliphatic spirocyclic ring system;
and/or Q⁴ may be methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl and an amino acyl residue of structural formula:

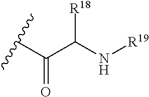

wherein R¹⁸ is hydrogen, C₁₋₅ alkyl or phenylC₀₋₂ alkyl; and R¹⁹ is hydrogen, C₁₋₄ alkyl, C₁₋₄ alkylsulfonyl or phenylC₁₋₂ alkylsulfonyl, or a group COR²⁰ wherein R²⁰ is C₁₋₄ alkyl optionally substituted by phenyl, C₁₋₄ alkoxy optionally substituted by phenyl, C₁₋₄alkylamino optionally substituted by C₁₋₄ alkyl optionally substituted by phenyl.

Suitably Q¹ is selected from hydrogen, monophosphate, diphosphate, or triphosphate, or C₁-C₁₆-alkylcarbonyl or a monophosphate prodrug of structure described before wherein: R⁷ is hydrogen, methyl or benzyl; more suitably hydrogen or methyl; R⁸ is hydrogen or methyl; more suitably hydrogen; R⁹ is Ph, CO₂R¹¹ or CR¹³R¹⁴OC(O)R¹² and R¹⁰ is hydroxyl or OR¹⁶; wherein R¹⁶ is an aromatic or heteroaromatic ring or CH₂CH₂SR¹⁷, where R¹⁷ is C₁-C₆ alkylcarbonyl, optionally substituted with a hydroxyl group; more suitably R¹⁰ is hydroxyl, O-phenyl or CH₂CH₂S—C₁-C₆-alkylcarbonyl optionally substituted with a hydroxyl group; most suitably R¹⁰ is hydroxyl or CH₂CH₂S S-tert-butylcarbonyl or CH₂CH₂S-hydroxy-tert-butylcarbonyl.

Suitably R¹¹ is C₁-C₁₆ alkyl, preferably C₇-C₁₆ alkyl; R¹² is C₁-C₁₆ alkyl, preferably C₇-C₁₆ alkyl; and R¹³ and R¹⁴ are both hydrogen.

Most suitably Q¹ is hydrogen or triphosphoryl.

Suitably Q² is selected from hydrogen, C₁-C₁₆-alkylcarbonyl or an amino acyl residue of the structure described before wherein R¹⁸ is hydrogen or C₁₀ alkyl, more suitably methyl, and R¹⁹ is hydrogen Most suitably Q² is hydrogen.

The compounds of formula (I) have the indicated stereochemical configuration.

Preferred embodiment the compound of the formula (I) include those compounds selected from the formula (III), (IV), (V), and (VI):

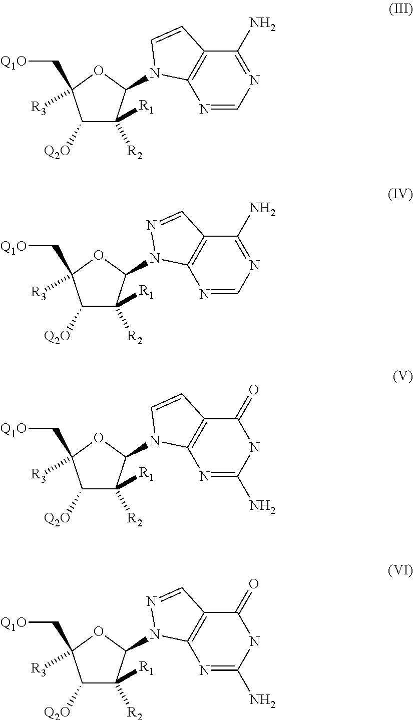

and pharmaceutically acceptable salts thereof; wherein R¹ to R⁶, Z, Q¹ and Q² are as hereinbefore defined.

Preferably the compound of the formula (I) is a compound of the formula (VII):

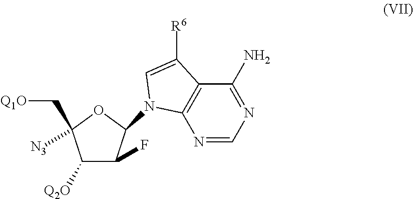

wherein R⁶, Q¹ and Q² are as hereinbefore defined and pharmaceutically acceptable salts thereof.

Preferably R⁶ is hydrogen. Preferably Q¹ and Q² are hydrogen.

The compounds of the formula (VII) are novel compounds and therefore form a further aspect of the present invention.

Preferred compounds of the present invention include:
7-(4-azido-2-deoxy-2-fluoro-β-D-arabinofuranosyl)-7H-pyrrolo[2,3d]pyrimidin-4-amine and pharmaceutically acceptable salts thereof.

The compounds of formula (I) are useful as inhibitors of RNA-dependent RNA viral polymerases and in particular of HCV NS5B polymerase. They are also inhibitors of RNA-dependent RNA viral replication and in particular of HCV replication and are useful for the treatment of RNA-dependent RNA viral infections and in particular for the treatment of HCV infection. The compounds of the formula (I) wherein Q¹ and Q² are other than 5′-triphosphate and hydroxyl respectively may act as prodrugs or may be converted into compounds of the formula (I) which are useful for the treatment of RNA-dependent RNA viral infection and in particular for the treatment of HCV infection.

Without limitation as to their mechanism of action, prodrugs of the compounds of the present invention as herein defined act as precursors of the corresponding nucleoside 5′-monophosphates. Endogenous kinase enzymes convert the 5′-monophosphates into their 5′-triphosphate derivatives which are the inhibitors of the RNA-dependent RNA viral polymerases. Thus, the prodrugs may provide for more efficient target cell penetration than the nucleoside itself, may be less susceptible to metabolic degradation, and may have the ability to target a specific tissue, such as the liver, resulting in a wider therapeutic index allowing for lowering the overall dose of the antiviral agent.

Also encompassed within the present invention are pharmaceutical compositions containing the compounds alone or in combination with other agents active against RNA-dependent RNA viruses and in particular against HCV as well as methods for the inhibition of RNA-dependent RNA viral replication and for the treatment of RNA-dependent RNA viral infections.

In one embodiment of the present invention, the compounds of the present invention are useful as precursors to inhibitors of positive-sense single-stranded RNA-dependent RNA viral polymerases, inhibitors of positive-sense single-stranded RNA-dependent RNA viral replication, and/or for the treatment of positive-sense single-stranded RNA-dependent RNA viral infections. In a class of this embodiment, the positive-sense single-stranded RNA-dependent RNA virus is a Flaviviridae virus or a Picornaviridae virus. In a subclass of this class, the Picornaviridae virus is a rhinovirus, a poliovirus, or a hepatitis A virus. In a second subclass of this class, the Flaviviridae virus is selected from the group consisting of hepatitis C virus, yellow fever virus, dengue virus, West Nile virus, Japanese encephalitis virus, Banzi virus, and bovine viral diarrhea virus (BVDV). In a subclass of this subclass, the Flaviviridae virus is hepatitis C virus.

Another aspect of the present invention is concerned with a method for inhibiting RNA-dependent RNA viral polymerases, a method for inhibiting RNA-dependent RNA viral replication, and/or a method for treating RNA-dependent RNA viral infections in a mammal in need thereof comprising administering to the mammal a therapeutically effective amount of a compound of structural formula (I).

In one embodiment of this aspect of the present invention, the RNA-dependent RNA viral polymerase is a positive-sense single-stranded RNA-dependent RNA viral polymerase. In a class of this embodiment, the positive-sense single-stranded RNA-dependent RNA viral polymerase is a Flaviviridae viral polymerase or a Picornaviridae viral polymerase. In a subclass of this class, the Picornaviridae viral polymerase is rhinovirus polymerase, poliovirus polymerase, or hepatitis A virus polymerase. In a second subclass of this class, the Flaviviridae viral polymerase is selected from the group consisting of hepatitis C virus polymerase, yellow fever virus polymerase, dengue virus polymerase, West Nile virus polymerase, Japanese encephalitis virus polymerase, Banzi virus polymerase, and bovine viral diarrhea virus (BVDV) polymerase. In a subclass of this subclass, the Flaviviridae viral polymerase is hepatitis C virus polymerase.

In a second embodiment of this aspect of the present invention, the RNA-dependent RNA viral replication is a positive-sense single-stranded RNA-dependent RNA viral replication. such as a Flaviviridae viral replication or Picornaviridae viral replication. In one subclass, the Picornaviridae viral replication is rhinovirus replication, poliovirus replication, or hepatitis A virus replication. In a second subclass, the Flaviviridae viral replication is selected from the group consisting of hepatitis C virus replication, yellow fever virus replication, dengue virus replication, West Nile virus replication, Japanese encephalitis virus replication, Banzi virus replication, and bovine viral diarrhea virus replication and preferably hepatitis C virus replication.

In a third embodiment of this aspect of the present invention, the RNA-dependent RNA viral infection is a positive-sense single-stranded RNA-dependent viral infection such as a Flaviviridae viral infection or Picornaviridae viral infection. In a subclass of this class, the Picornaviridae viral infection is rhinovirus infection, poliovirus infection, or hepatitis A virus infection. In a second subclass of this class, the Flaviviridae viral infection is selected from the group consisting of hepatitis C virus infection, yellow fever virus infection, dengue virus infection, West Nile virus infection, Japanese encephalitis virus infection, Banzi virus infection, and bovine viral diarrhea virus infection. Preferably, the Flaviviridae viral infection is hepatitis C virus infection.

Throughout the instant application, the following terms have the indicated meanings:

The term “aliphatic” shall mean alkyl, alkenyl and alkynyl groups containing the designated number of carbon atoms.

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, heptyl, 1-propylbutyl, octyl, 2-propylpentyl, and the like.

The term “adamantyl” encompasses both 1-adamantyl and 2-adamantyl.

The term “alkenyl” shall mean straight or branched chain alkenes containing the designated number of carbon atoms, or any number within this range (e.g., ethenyl, propenyl, butenyl, pentenyl, oleyl, etc.).

The term “alkynyl” shall mean straight or branched chain alkynes containing the designated number of carbon atoms, or any number within this range (e.g., ethynyl, propynyl, etc.).

The term “cycloalkyl” shall mean cyclic rings of alkanes having the designated number of carbon atoms, or any number within this range (examples include cyclopropyl, cyclobutyl, cyclopentyl, cyclohexyl, cycloheptyl, or cyclooctyl).

The term “cycloalkenyl” shall mean cyclic rings of alkenes having the designated number of carbon atoms, or any number within this range (i.e., cyclopropenyl, cyclobutenyl, cyclopentenyl, cyclohexenyl, cycloheptenyl, or cyclooctenyl).

The term “C₁₋₆ aliphatic group” refers to alkyl, alkenyl, alkynyl, cycloalkyl, cycloalkenyl or cycloalkynyl groups that contain from one to six carbon atoms.

The term “alkoxy” refers to straight or branched chain alkoxides of the number of carbon atoms specified (e.g., C₁₋₄alkoxy), or any number within this range [i.e., methoxy, ethoxy, isopropoxy, etc.].

The term “alkylamino” refers to straight or branched alkylamines of the number of carbon atoms specified (e.g., C₁₋₄alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆alkylsulfonyl), or any number within this range [i.e., methylsulfonyl (MeSO₂—), ethylsulfonyl, isopropylsulfonyl, etc.].

The term “alkyloxycarbonyl” refers to straight or branched chain esters of a carboxylic acid or carbamic acid group present in a compound of the present invention having the number of carbon atoms specified (e.g., C₁₋₈alkyloxycarbonyl), or any number within this range [i.e., methyloxycarbonyl (MeOCO—), ethyloxycarbonyl, or butyloxycarbonyl].

The term “alkylcarbonyl” refers to straight or branched chain alkyl acyl group of the specified number of carbon atoms (e.g., C₁₋₈alkylcarbonyl), or any number within this range [i.e., methyloxycarbonyl (MeOCO—), ethyloxycarbonyl, or butyloxycarbonyl].

The term “halo” is intended to include fluoro, chloro, bromo and iodo [i.e. chloro or fluoro].

The term “monophosphate” refers to —P(O)(OH)₂, The term “diphosphate” refers to the radical having the structure:

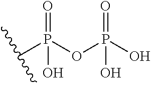

and the term “triphosphate” refers to the radical having the structure:

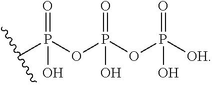

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally.

The term “5′-triphosphate” refers to a triphosphoric acid ester derivative of the 5′-hydroxyl group of a nucleoside compound of the present invention having the following general structural formula

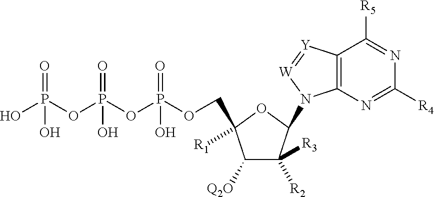

wherein R¹, R², R³, R⁴, R⁵, Y, W, Q¹ and Q² are as defined above.

The term “composition”, as in “pharmaceutical composition,” is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The terms “administration of” and “administering a” compound should be understood to mean providing a compound of the invention or a prodrug of a compound of the invention to the individual in need.

Another aspect of the present invention is concerned with a method of inhibiting HCV NS5B polymerase, inhibiting HCV replication, or treating HCV infection with a compound of the present invention in combination with one or more agents useful for treating HCV infection. Such agents active against HCV include, but are not limited to, ribavirin, levovirin, viramidine, nitazoxanide, thymosin alpha-1, interferon-β, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), pegylated interferon-α2a (Pegasys™), interferon-α2b (such as Intron-A interferon available from Schering Corp., Kenilworth, N.J.), pegylated interferon-α2b (PegIntron™), a recombinant consensus interferon (such as interferon alphacon-1), and a purified interferon-α product. Amgen's recombinant consensus interferon has the brand name Infergen®. Levovirin is the L-enantiomer of ribavirin which has shown immunomodulatory activity similar to ribavirin. Viramidine represents an analog of ribavirin disclosed in WO 01/60379 (assigned to ICN Pharmaceuticals). In accordance with this method of the present invention, the individual components of the combination can be administered separately at different times during the course of therapy or concurrently in divided or single combination forms. The instant invention is therefore to be understood as embracing all such regimes of simultaneous or alternating treatment, and the term “administering” is to be interpreted accordingly. It will be understood that the scope of combinations of the compounds of this invention with other agents useful for treating HCV infection includes in principle any combination with any pharmaceutical composition for treating HCV infection. When a compound of the present invention or a pharmaceutically acceptable salt thereof is used in combination with a second therapeutic agent active against HCV, the dose of each compound may be either the same as or different from the dose when the compound is used alone.

For the treatment of HCV infection, the compounds of the present invention may also be administered in combination with an agent that is an inhibitor of HCV NS3 serine protease. HCV NS3 serine protease is an essential viral enzyme and has been described to be an excellent target for inhibition of HCV replication. Both substrate and non-substrate based inhibitors of HCV NS3 protease inhibitors are disclosed in WO 98/22496, WO 98/46630, WO 99/07733, WO 99/07734, WO 99/38888, WO 99/50230, WO 99/64442, WO 00/09543, WO 00/59929, GB-2337262, WO 02/18369, WO 02/08244, WO 02/48116, WO 02/48172, WO 05/037214, and U.S. Pat. No. 6,323,180. HCV NS3 protease as a target for the development of inhibitors of HCV replication and for the treatment of HCV infection is discussed in B. W. Dymock, “Emerging therapies for hepatitis C virus infection,” Emerging Drugs, 6: 13-42 (2001). Specific HCV NS3 protease inhibitors combinable with the compounds of the present invention include BILN2061, VX-950, SCH6, SCH7, and SCH-503034.

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of HCV replication. Therefore, the compounds of the present invention may also be administered in combination with an inhibitor of IMPDH, such as VX-497, which is disclosed in WO 97/41211 and WO 01/00622 (assigned to Vertex); another IMPDH inhibitor, such as that disclosed in WO 00/25780 (assigned to Bristol-Myers Squibb); or mycophenolate mofetil [see A. C. Allison and E. M. Eugui, Agents Action, 44 (Suppl.): 165 (1993)].

For the treatment of HCV infection, the compounds of the present invention may also be administered in combination with the antiviral agent amantadine (1-aminoadamantane) [for a comprehensive description of this agent, see J. Kirschbaum, Anal. Profiles Drug Subs. 12: 1-36 (1983)].

The compounds of the present invention may also be combined for the treatment of HCV infection with antiviral 2′-C-branched ribonucleosides disclosed in R. E. Harry-O'kuru, et al., J. Org. Chem., 62: 1754-1759 (1997); M. S. Wolfe, et al., Tetrahedron Lett., 36: 7611-7614 (1995); U.S. Pat. No. 3,480,613 (Nov. 25, 1969); U.S. Pat. No. 6,777,395 (Aug. 17, 2004); U.S. Pat. No. 6,914,054 (Jul. 5, 2005); International Publication Numbers WO 01/90121 (29 Nov. 2001); WO 01/92282 (6 Dec. 2001); WO 02/32920 (25 Apr. 2002); WO 02/057287 (25 Jul. 2002); WO 02/057425 (25 Jul. 2002); WO 04/002422 (8 Jan. 2004); WO 04/002999 (8 Jan. 2004); WO 04/003000 (8 Jan. 2004); WO 04/002422 (8 Jan. 2004); US Patent Application Publications 2005/0107312; US 2005/0090463; US 2004/0147464; and US 2004/0063658; the contents of each of which are incorporated by reference in their entirety. Such 2′-C-branched ribonucleosides include, but are not limited to, 2′-C-methylcytidine, 2′-fluoro-2′-C-methylcytidine, 2′-C-methyluridine, 2′-C-methyladenosine, 2′-C-methylguanosine, and 9-(2-C-methyl-β-D-ribofuranosyl)-2,6-diaminopurine; the corresponding amino acid esters of the furanose C-2′, C-3′, and C-5′ hydroxyls (such as 3′-O-(L-valyl)-2′-C-methylcytidine dihydrochloride, also referred to as valopicitabine dihydrochloride or NM-283 and 3′-O-(L-valyl)-2′-fluoro-2′-C-methylcytidine), and the corresponding optionally substituted cyclic 1,3-propanediol esters of their 5′-phosphate derivatives.

The compounds of the present invention may also be combined for the treatment of HCV infection with other nucleosides having anti-HCV properties, such as those disclosed in U.S. Pat. No. 6,864,244 (Mar. 8, 2005); WO 02/51425 (4 Jul. 2002), assigned to Mitsubishi Pharma Corp.; WO 01/79246, WO 02/32920, and WO 02/48165 (20 Jun. 2002), assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), assigned to Hoffmann-LaRoche; U.S. 2002/0019363 (14 Feb. 2002); WO 02/100415 (19 Dec. 2002); WO 03/026589 (3 Apr. 2003); WO 03/026675 (3 Apr. 2003); WO 03/093290 (13 Nov. 2003): US 2003/0236216 (25 Dec. 2003); US 2004/0006007 (8 Jan. 2004); WO 04/011478 (5 Feb. 2004); WO 04/013300 (12 Feb. 2004); US 2004/0063658 (1 Apr. 2004); and WO 04/028481 (8 Apr. 2004).

In one embodiment, nucleoside HCV NS5B polymerase inhibitors that may be combined with the nucleoside derivatives of the present invention are selected from the following compounds: 4′-azido-cytidine; 4-amino-7-(2-C-methyl-β-D-ribofuranosyl)-7H-pyrrolo[2,3-c]pyrimidine; 4-amino-7-(2-C-hydroxymethyl-β-D-ribofuranosyl)-7H-pyrrolo[2,3-d]pyrimidine; 4-amino-7-(2-C-fluoromethyl-β-D-ribofuranosyl)-7H-pyrrolo[2,3-d]pyrimidine; 4-amino-5-fluoro-7-(2-C-methyl-β-D-ribofuranosyl)-7H-pyrrolo[2,3-d]pyrimidine; 2-amino-7-(2-C-methyl-β-D-ribofuranosyl)-7H-pyrrolo[2,3-d]pyrimidin-4(3H)-one; 4-amino-7-(2-C,2-O-dimethyl-β-D-ribofuranosyl)-7H-pyrrolo[2,3-d]pyrimidine; β-D-2′-deoxy-2′-fluoro-2′-C-methyl-cytidine and pharmaceutically acceptable salts and prodrugs thereof.

The compounds of the present invention may also be combined for the treatment of HCV infection with non-nucleoside inhibitors of HCV polymerase such as those disclosed in WO 01/77091 (18 Oct. 2001), assigned to Tularik, Inc.; WO 01/47883 (5 Jul. 2001), assigned to Japan Tobacco, Inc.; WO 02/04425 (17 Jan. 2002), assigned to Boehringer Ingelheim; WO 02/06246 (24 Jan. 2002), assigned to Istituto di Ricerche di Biologia Molecolare P. Angeletti S.p.A.; WO 02/20497 (3 Mar. 2002); WO 2005/016927 (in particular JTK003), assigned to Japan Tobacco, Inc.; the contents of each of which are incorporated herein by reference in their entirety; and HCV-796 (Viropharma Inc.).

In one embodiment, non-nucleoside HCV NS5B polymerase inhibitors that may be combined with the nucleoside derivatives of the present invention are selected from the following compounds: 14-cyclohexyl-6-[2-(dimethylamino)ethyl]-7-oxo-5,6,7,8-tetrahydroindolo[2,1-a][2,5]benzodiazocine-11-carboxylic acid; 14-cyclohexyl-6-(2-morpholin-4-ylethyl)-5,6,7,8-tetrahydroindolo[2,1-a][2,5]benzodiazocine-11-carboxylic acid; 14-cyclohexyl-6-[2-(dimethylamino)ethyl]-3-methoxy-5,6,7,8-tetrahydroindolo[2,1-a][2,5]benzodiazocine-11-carboxylic acid; 14-cyclohexyl-3-methoxy-6-methyl-5,6,7,8-tetrahydroindolo[2,1-a][2,5]benzodiazocine-11-carboxylic acid; methyl ({[(14-cyclohexyl-3-methoxy-6-methyl-5,6,7,8-tetrahydroindolo[2,1-a][2,5]benzodiazocin-11-yl)carbonyl]amino}sulfonyl)acetate; ({[(14-cyclohexyl-3-methoxy-6-methyl-5,6,7,8-tetrahydroindolo[2,1-a][2,5]benzodiazocin-11-yl)carbonyl]amino}sulfonyl)acetic acid; 14-cyclohexyl-N-[(dimethylamino)sulfonyl]-3-methoxy-6-methyl-5,6,7,8-tetrahydroindolo[2,1-a][2,5]benzodiazocine-11-carboxamide; 3-chloro-14-cyclohexyl-6-[2-(dimethylamino)ethyl]-7-oxo-5,6,7,8-tetrahydroindolo[2,1-a][2,5]benzodiazocine 11-carboxylic acid; N-(11-carboxy-14-cyclohexyl-7,8-dihydro-6H-indolo[1,2-e][1,5]benzoxazocin-7-yl)-N,N-dimethylethane-1,2-diaminium bis(trifluoroacetate); 14-cyclohexyl-7,8-dihydro-6H-indolo[1,2-e][1,5]benzoxazocine-11-carboxylic acid; 14-cyclohexyl-6-methyl-7-oxo-5,6,7,8-tetrahydroindolo[2,1-a][2,5]benzodiazocine-11-carboxylic acid; 14-cyclo hexyl-3-methoxy-6-methyl-7-oxo-5,6,7,8-tetrahydroindolo[2,1-a][2,5]benzodiazocine-11-carboxylic acid; 14-cyclohexyl-6-[2-(dimethylamino)ethyl]-3-methoxy-7-oxo-5,6,7,8-tetrahydroindolo[2,1-a][2,5]benzodiazocine-11-carboxylic acid; 14-cyclohexyl-6-[3-(dimethylamino)propyl]-7-oxo-5,6,7,8-tetrahydroindolo[2,1-a][2,5]benzodiazocine-11-carboxylic acid; 14-cyclohexyl-7-oxo-6-(2-piperidin-1-ylethyl)-5,6,7,8-tetrahydroindolo[2,1-a][2,5]benzodiazocine-11-carboxylic acid; 14-cyclohexyl-6-(2-morpholin-4-ylethyl)-7-oxo-5,6,7,8-tetrahydroindolo[2,1-a][2,5]benzodiazocine-11-carboxylic acid; 14-cyclohexyl-6-[2-(diethylamino)ethyl]-7-oxo-5,6,7,8-tetrahydroindolo[2,1-a][2,5]benzodiazocine-11-carboxylic acid; 14-cyclohexyl-6-(1-methylpiperidin-4-yl)-7-oxo-5,6,7,8-tetrahydroindolo[2,1-a][2,5]benzodiazocine-11-carboxylic acid; 14-cyclohexyl-N-[(dimethylamino)sulfonyl]-7-oxo-6-(2-piperidin-1-ylethyl)-5,6,7,8-tetrahydroindolo[2,1-a][2,5]benzodiazocine-11-carboxamide; 14-cyclohexyl-6-[2-(dimethylamino)ethyl]-N-[(dimethylamino)sulfonyl]-7-oxo-5,6,7,8-tetrahydroindolo[2,1-a][2,5]benzodiazocine-11-carboxamide; 14-cyclopentyl-6-[2-(dimethylamino)ethyl]-7-oxo-5,6,7,8-tetrahydroindolo[2,1-a][2,5]benzodiazocine-11-carboxylic acid; 14-cyclohexyl-5,6,7,8-tetrahydroindolo[2,1-a][2,5]benzodiazocine-11-carboxylic acid; 6-allyl-14-cyclohexyl-3-methoxy-5,6,7,8-tetrahydroindolo[2,1-a][2,5]benzodiazocine-11-carboxylic acid; 14-cyclopentyl-6-[2-(dimethylamino)ethyl]-5,6,7,8-tetrahydroindolo[2,1-a][2,5]benzodiazocine-11-carboxylic acid; 14-cyclohexyl-6-[2-(dimethylamino)ethyl]-5,6,7,8-tetrahydroindolo[2,1-a][2,5]benzodiazocine-11-carboxylic acid; 13-cyclohexyl-5-methyl-4,5,6,7-tetrahydrofuro[3′,2′:6,7][1,4]diazocino[1,8-a]indole-10-carboxylic acid; 15-cyclohexyl-6-[2-(dimethylamino)ethyl]-7-oxo-6,7,8,9-tetrahydro-5H-indolo[2,1-a][2,6]benzodiazonine-12-carboxylic acid; 15-cyclohexyl-8-oxo-6,7,8,9-tetrahydro-5H-indolo[2,1-a][2,5]benzodiazonine-12-carboxylic acid; 13-cyclohexyl-6-oxo-6,7-dihydro-5H-indolo[1,2-d][1,4]benzodiazepine-10-carboxylic acid; and pharmaceutically acceptable salts thereof.

By “pharmaceutically acceptable” is meant that the carrier, diluent, or excipient must be compatible with the other ingredients of the formulation and not deleterious to the recipient thereof.

Also included within the present invention are pharmaceutical compositions comprising the compounds of the present invention in association with a pharmaceutically acceptable carrier. Another example of the invention is a pharmaceutical composition made by combining any of the compounds described above and a pharmaceutically acceptable carrier. Another illustration of the invention is a process for making a pharmaceutical composition comprising combining any of the compounds described above and a pharmaceutically acceptable carrier.

Also included within the present invention are pharmaceutical compositions useful for inhibiting RNA-dependent RNA viral polymerases in particular HCV NS5B polymerase comprising an effective amount of a compound of the present invention and a pharmaceutically acceptable carrier. Pharmaceutical compositions useful for treating RNA-dependent RNA viral infections in particular HCV infection are also encompassed by the present invention as well as a method of inhibiting RNA-dependent RNA viral polymerases in particular HCV NS5B polymerase and a method of treating RNA-dependent viral replication and in particular HCV replication. Additionally, the present invention is directed to a pharmaceutical composition comprising a therapeutically effective amount of a compound of the present invention in combination with a therapeutically effective amount of another agent active against RNA-dependent RNA viruses and in particular against HCV. Agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), interferon-α2b (such as Intron-A interferon available from Schering Corp., Kenilworth, N.J.), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics, and Therapeutic Potential,” Ann. Rep. Med. Chem., 35: 201-210 (2000).

Another aspect of the present invention provides for the use of the compounds of the present invention and their pharmaceutical compositions for the manufacture of a medicament for the inhibition of RNA-dependent RNA viral replication, in particular HCV replication, and/or the treatment of RNA-dependent RNA viral infections, in particular HCV infection. Yet a further aspect of the present invention provides for the compounds of the present invention and their pharmaceutical compositions for use as a medicament for the inhibition of RNA-dependent RNA viral replication, in particular HCV replication, and/or for the treatment of RNA-dependent RNA viral infections, in particular HCV infection.

The pharmaceutical compositions of the present invention comprise a compound of formula (I) as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably, compounds of structural formula I are administered orally. Also preferably, compounds of structural formula I are administered parenterally.

For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions are preferably provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

The effective dosage of active ingredient employed may vary depending on the particular compound employed, the mode of administration, the condition being treated and the severity of the condition being treated. Such dosage may be ascertained readily by a person skilled in the art. This dosage regimen may be adjusted to provide the optimal therapeutic response.

The compounds of the present invention contain one or more asymmetric centers and can thus occur as racemates and racemic mixtures, single enantiomers, diastereoisomeric mixtures and individual diastereoisomers. When R¹⁸ in the amino acyl residue embodiment of Q² is a substituent other than hydrogen in the formula

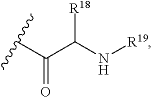

the amino acyl residue contains an asymmetric center and is intended to include the individual R- and S-stereoisomers as well as RS-diastereoisomeric mixtures. In one embodiment, the stereochemistry at the stereogenic carbon corresponds to that of an S-amino acid, that is, the naturally occurring alpha-amino acid stereochemistry, as depicted in the formula:

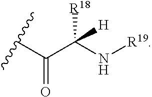

Furthermore, when R⁹ is:

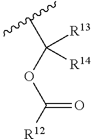

and R¹³ and R¹⁴ are not both hydrogen, the carboxy residue contains an asymmetric center and is intended to include the individual R- and S-stereoisomers as well as RS-stereoisomeric mixtures. Thus, when R⁴ and R⁵ are also not both hydrogen, the aminoalcohol residue contains two asymmetric centers and is intended to include the individual R,R-, R,S-, S,R- and S,S-diastereoisomers as well as mixtures thereof.

The present invention is meant to comprehend compounds having the β-D stereochemical configuration for the five-membered furanose ring as depicted in the structural formula, that is, nucleoside phosphoramidates in which the substituents at C-1 and C-4 of the five-membered furanose ring have the β-stereochemical configuration (“up” orientation as denoted by a bold line). Some of the compounds described herein contain olefinic double bonds, and unless specified otherwise, are meant to include both E and Z geometric isomers.

Some of the compounds described herein may exist as tautomers such as keto-enol tautomers. The individual tautomers as well as mixtures thereof are encompassed with compounds of structural formula (I).

Compounds of structural formula (I) may be separated into their individual diastereoisomers by, for example, fractional crystallization from a suitable solvent, for example methanol or ethyl acetate or a mixture thereof, or via chiral chromatography using an optically active stationary phase.

Alternatively, any stereoisomer of a compound of the structural formula (I) may be obtained by stereospecific synthesis using optically pure starting materials or reagents of known configuration.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or hydroxyl group being present in the compounds of the present invention, pharmaceutically acceptable prodrug esters of carboxylic acid derivatives, such as methyl, ethyl, or pivaloyloxymethyl esters or prodrug acyl derivatives of the ribose C-2′, C-3′, and C-5′ hydroxyls, such as O-acetyl, O-pivaloyl, O-benzoyl and O-aminoacyl, can be employed. Included are those esters and acyl groups known in the art for modifying the bioavailability, tissue distribution, solubility, and hydrolysis characteristics for use as sustained-release or prodrug formulations. The contemplated derivatives are readily convertible in vivo into the required compound. Thus, in the methods of treatment of the present invention, the terms “administering” and “administration” is meant to encompass the treatment of the viral infections described with a compound specifically disclosed or with a compound which may not be specifically disclosed, but which converts to the specified compound in vivo after administration to the mammal, including a human patient. Conventional procedures for the selection and preparation of suitable prodrug derivatives are described, for example, in “Design of Prodrugs,” ed. H. Bundgaard, Elsevier, 1985, which is incorporated by reference herein in its entirety.

### General Description of Synthesis

The compounds described in the present invention may be prepared as outlined in Scheme 1. A sugar building block such as 1, suitably protected and bearing a leaving group in the anomeric position (for example 3,5-di-O-benzoyl-2-deoxy-2-fluoro-α-D-arabinofuranosyl bromide, prepared as reported in J. Org. Chem. 1988, 53, 85) can be reacted in the presence of an appropriate base with a functionalized heterocyclic derivative such as 2. Removal of the protecting groups is then necessary to allow further manipulation of the 5′-hydroxyl moiety. Typically, conversion of the 5′-OH into the corresponding 5′-iodide is achieved by standard methodologies known to those skilled in the art: suitable procedures include treatment of 3 with iodine and Ph₃P in pyridine, optionally in combination with a suitable organic solvent such as dioxane, acetonitrile or similar, or alternatively reaction of 3 with suitable reagents such as methyltriphenoxyphosphonium iodide. Treatment of the resulting iodide with an appropriate organic base, for example DBU or ^{t}BuOK, and subsequent introduction of suitable protecting groups on the hydroxyl and amino moieties gives the required 4′,5′-olefin 4. The desired 4′-azido functionality is then installed via a two-step sequence featuring olefin epoxidation followed by Lewis acid promoted azide addition. A practical way to perform this transformation relies on the use of DMDO for the epoxidation step and treatment with TMS-N₃ in the presence of SnCl₄, as described by McGuigan et al., J. Med. Chem. 2007, 50, 54623. Final deprotection and purification steps (typically by preparative HPLC) complete the synthetic sequence towards the compounds described in this invention.

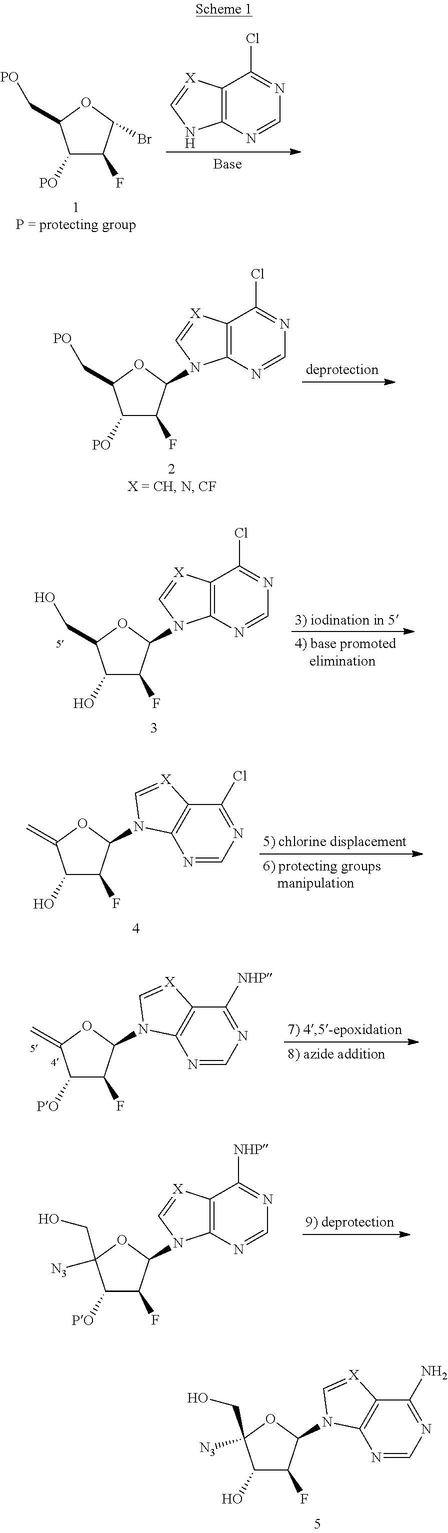

In selected cases, it can be advantageous to modify slightly the order of the synthetic steps, as depicted in Scheme 2. In particular, it might be beneficial to perform the chlorine displacement step before proceeding with the introduction of the iodine in 5′-position.

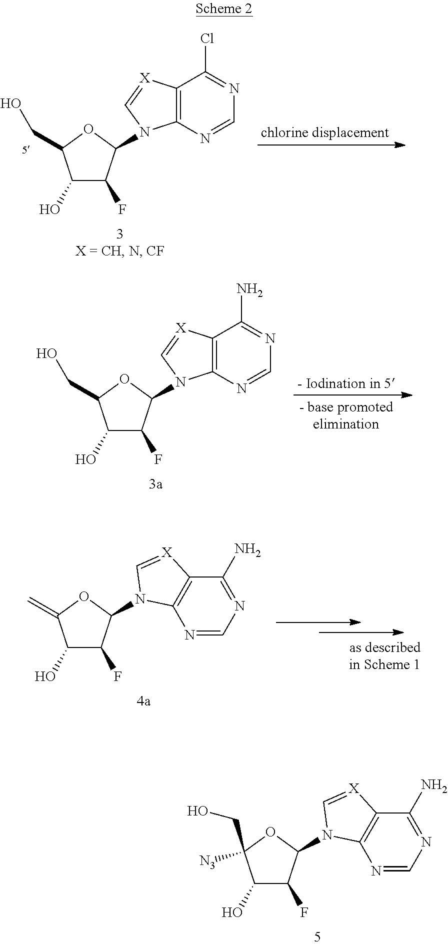

Employing methods known to those skilled in the art, the nucleoside analogues herein described can be converted into a variety of nucleotide derivatives such as the corresponding monophophates and monophosphate prodrugs, diphosphates and triphosphates (selected references: Ludwig, J. Acta Biochim. Biophys. Acad. Sci. Hung. 1981, 16, 131; Ludwig, J., Eckstein, F. J. Org. Chem. 1989, 54, 613; Mishra, N. C.; Broom, A. D. J. Chem. Soc., Chem. Commun. 1991, 1276; McGuigan et al., J. Med. Chem. 1993, 36, 1048; Uchiyama et al., J. Org. Chem., 1993, 58, 373). Non limiting examples of the methodologies employed for the preparation of triphosphate derivatives are described in Scheme 3.

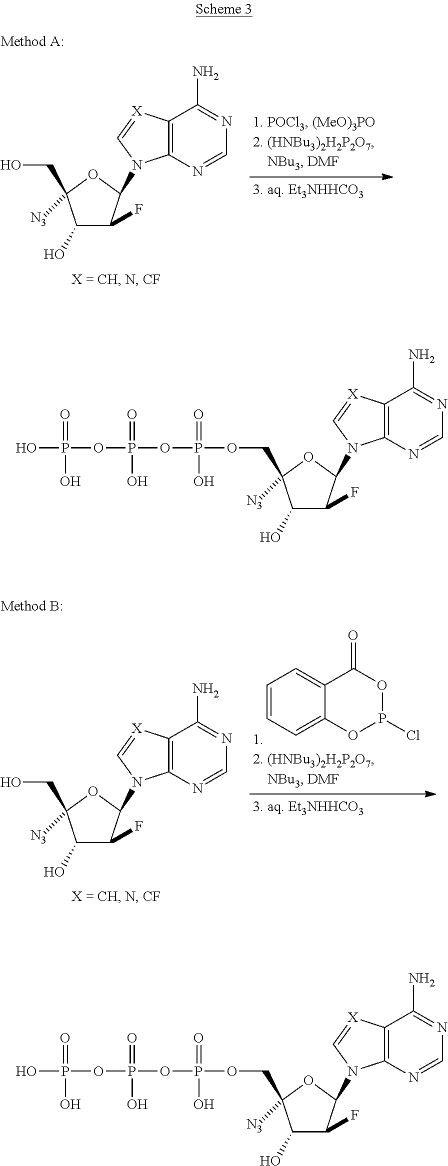

Non limiting examples of the synthesis of nucleoside monophosphates and nucleoside monophosphate prodrugs are depicted in Scheme 4.

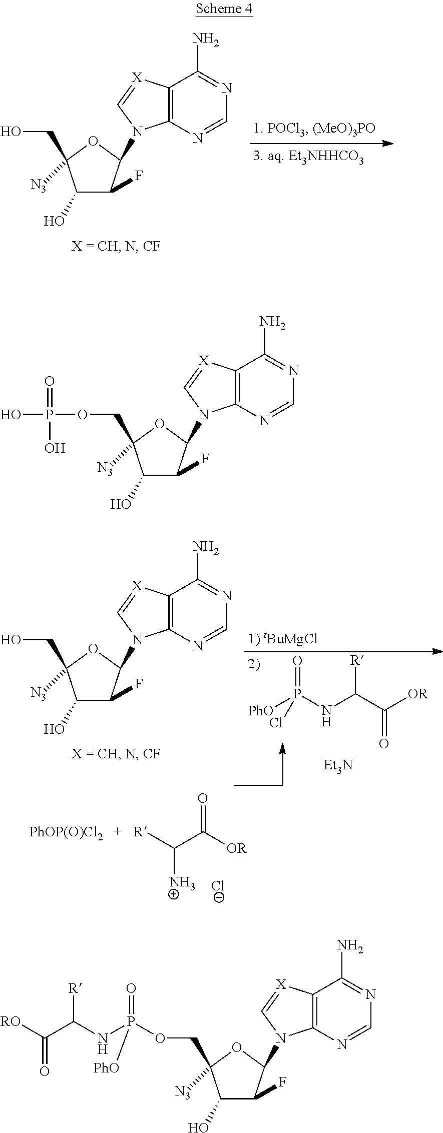

### General Synthetic Procedures

All solvents were obtained from commercial sources and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out either on silica gel following published procedure (W. C. Still et al., J. Org. Chem. 1978, 43, 2923) or on semi-automated flash chromatography systems utilizing pre-packed columns.

Reagents were usually obtained directly from commercial suppliers (and used as supplied) but a limited number of compounds from in-house corporate collections were utilised. In the latter case the reagents are readily accessible using routine synthetic steps that are either reported in the scientific literature or are known to those skilled in the art.

¹H, ¹⁹F and ³¹P nmr spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; br, broad, and combinations thereof); coupling constant(s) in hertz; number of protons. Mass spectral (MS) data were obtained on Waters Micromass ZMD, operating in negative (ES⁻) or positive (ES⁺) ionization mode and results are reported as the ratio of mass over charge (m/z). Preparative scale RP-HPLC separations were carried out on: 1) Waters Delta Prep 4000 preparative chromatograpy system, equipped with a Waters 2487 Dual λ absorbance detector; 2) Automated (UV-triggered) RP-HPLC Shimadzu Discovery VP system, incorporating an LC-8A preparative liquid chromatography module, an SPD-10A UV-VIS detector and a FRC-10A fraction collector module. In both cases the stationary phase employed was an Atlantis Prep T3 5 μm OBD (19×150 mm) or a XBridge Prep C₁₈ 5 μm OBD (19×150 mm). Unless otherwise stated, the mobile phase comprised a linear gradient of binary mixture of MeCN (containing 0.1% TFA) and water (containing 0.1% TFA), or MeCN and 5 mM dimethylhexylammonium bicarbonate in water using flow rates between 15 and 25 mL/min.

Reactions under microwave irradiation were carried out in Emrys Optimizer reactor from Personal Chemistry, Sweden.

The following abbreviations are used in the Schemes and Examples:

AcOH: acetic acid; aq.: aqueous; bs: broad singlet; bt: broad triplet; DBU: 1,8-Diazabicyclo[5.4.0]undec-7-ene; DIAD: diisopropyl azodicarboxylate; DIPEA: diisopropylethyl amine; DMF: dimethylformamide; DMSO: dimethylsulfoxide; eq.: equivalent(s); Et₂O: diethyl ether; EtOAc: ethyl acetate; EtOH: ethanol; (HNBu₃)₂H₂P₂O₇: bis tributylammonium pyrophosphate; h: hour(s); M: molar; MeCN: acetonitrile; MeOH: methanol; (MeO)₃PO: trimethyl phosphate; min: minutes; NaBH₃CN: sodium cyanoborohydride; NBu₃: tributylamine; NMP: 1-methyl-2-pyrrolidinone; Pd(PPh₃)₄: tetrakis(triphenylphosphine)palladium (0); PE: petroleum ether; P(O)Cl₃: phosphorous oxychloride; RP-HPLC: reversed phase high-performance liquid chromatography; RT: room temperature; SPE: solid phase extraction; TBDMS: tert-butyldimethylsilyl; TEA: triethylamine; TFA: trifluoroacetic acid; THF: tetrahydrofuran; HPLC: ultra performance liquid chromatography.

### EXAMPLE 1

### (Table 1, entry 1): 7-(4-azido-2-deoxy-2-fluoro-β-D-arabinofuranosyl)-7H-pyrrolo[2,3-d]pyrimidin-4-amine

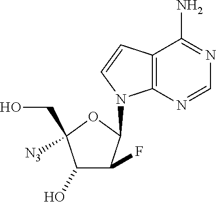

### Step 1: 4-chloro-7-(3,5-di-O-benzoyl-2-deoxy-2-fluoro-β-D-arabinofuranosyl)-7H-pyrrolo[2,3-d]pyrimidine

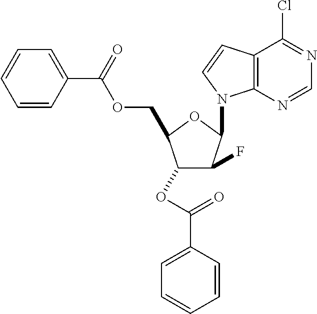

Tris(dioxa-3,6-heptyl)amine (0.2 eq) was added to a stirred suspension of KOH (3.0 eq) in dry AcCN (0.1 M) and the mixture was stirred at RT for 20 min. 4-Chloro-7H-pyrrolo[2,3-d]pyrimidine (1.0 eq) was then added in one portion and the resulting mixture was stirred at RT for 1 h. A 0.2 M solution of 3,5-di-O-benzoyl-2-deoxy-2-fluoro-α-D-arabinofuranosyl bromide in dry AcCN (1.0 eq; prepared as reported in J. Org. Chem. 1988, 53, 85) was added dropwise to the previous solution. The reaction mixture was stirred overnight at RT and then diluted with AcOEt. The brownish slurry was filtered over a short pad of Celite and washed with AcOEt. The organic phase was concentrated under reduced pressure and the residue purified by SiO₂ gel chromatography (gradient elution 10% to 40% AcOEt/PE) to give the title compound was as white foam (50%). ¹H-NMR (400 MHz, CDCl₃) δ 8.65 (s, 1H), 8.13-8.07 (m, 4H), 7.66-7.44 (m, 7H), 6.86 (m, 1H), 6.62 (m, 1H), 5.71 (m, 1H), 5.32 (m, 1H), 4.82-4.50 (m, 3H); ¹⁹F-NMR (400 MHz, CD₃Cl) δ−198.60; MS (ES⁺) C₂₅H₁₉ClFN₃O₅ requires: 495. found: 496 (M+H⁺).

### Step 2: 4-chloro-7-(2-deoxy-2-fluoro-β-D-arabinofuranosyl)-7H-pyrrolo[2,3-d]pyrimidine

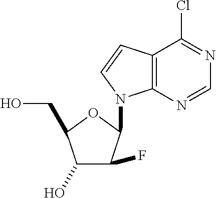

4-chloro-7-(3,5-di-O-benzoyl-2-deoxy-2-fluoro-β-D-arabinofuranosyl)-7H-pyrrolo[2,3-d]pyrimidine from Step 1 was dissolved in 7 N NH₃ in MeOH and the resulting solution (0.5 M) stirred overnight at RT. The volatiles were removed under reduced pressure and the title compound was crystallized from DCM/MeOH to a white powder (68%). ¹H-NMR (400 MHz, D₂O) δ 8.55 (s, 1H), 7.72 (s, 1H), 6.79-6.68 (m, 2H), 5.26 (m, 1H), 4.49 (m, 1H), 4.09-3.81 (m, 3H); MS (ES⁺) C₁₁H₁₁ClFN₃O₃ requires: 287. found: 288 (M+H⁺).

### Step 3: 7-(2,5-dideoxy-2-fluoro-β-D-threo-pent-4-enofuranosyl)-7H-pyrrolo[2,3-d]pyrimidin-4-amine

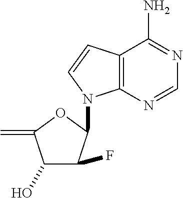

To a 0.1 M solution of 4-chloro-7-(2-deoxy-2-fluoro-β-D-arabinofuranosyl)-7H-pyrrolo[2,3-d]pyrimidine from Step 2 in pyridine were added iodine (1.5 eq) and Ph₃P (1.5 eq) and the resulting mixture was stirred overnight at RT. The volatiles were removed under reduced pressure, the residue co-evaporated twice with toluene and purified by flash chromatography eluting with DCM/MeOH to give 4-chloro-7-(2,5-dideoxy-2-fluoro-5-iodo-β-D-arabinofuranosyl)-7H-pyrrolo[2,3-d]pyrimidine as a pale yellow foam. The latter compound was dissolved in dry AcCN (0.1 M) and DBU (2.0 eq) was added. The reaction mixture was stirred overnight at RT and the volatiles were removed under reduced pressure. The residue was dissolved in 7 N NH₃ in MeOH and the resulting solution (0.1 M) was stirred overnight at 110° C. in a pressure sealed tube. The reaction mixture was allowed to cool to RT, the volatiles were removed under educed pressure and the title compound was crystallized from DCM/MeOH as white powder (16% over three steps). ¹H-NMR (400 MHz, CD₃CN/D₂O) δ 8.18 (s, 1H), 7.19 (m, 1H), 6.89 (dd, J₁ 18.0, J₂ 3.6, 1H), 6.61 (d, J 3.6, 1H), 5.16 (m, 1H), 4.90 (m, 1H), 4.58 (bs, 1H), 4.42 (bs, 1H); MS (ES⁺) C₁₁H₁₁FN₄O₂ requires: 250. found: 251 (M+H⁺).

### Step 4: 7-{3-O-[tert-butyl(dimethyl)silyl]-2,5-dideoxy-2-fluoro-β-D-threo-pent-4-enofuranosyl}-N-(2,2-dimethylpropanoyl)-7H-pyrrolo[2,3-d]pyrimidin-4-amine

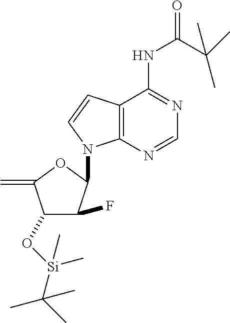

To a 0.2 M solution of 7-(2,5-dideoxy-2-fluoro-β-D-threo-pent-4-enofuranosyl)-7H-pyrrolo[2,3-d]pyrimidin-4-amine from Step 4 in dry Pyridine were added imidazole (8.0 eq) and TBDMS-Cl (4.0 eq) and the resulting mixture was stirred overnight at RT. The reaction was quenched with MeOH and the volatiles were removed under reduced pressure. The residue was dissolved in DCM, washed with citric acid (1 N aq. solution), water and brine. The organic phase was separated, dried over MgSO₄ and concentrated under reduced pressure. The residue was dissolved in dry DMF (0.1 N), DIPEA (2.0 eq) was added followed by pivaloyl chloride (1.9 eq) and the resulting reaction mixture was stirred overnight at RT. The volatiles were then removed under reduced pressure and the residue was purified by SiO₂ gel chromatography eluting with 2% MeOH/DCM to give the title compound as off-white foam. MS (ES⁺) C₂₂H₃₃FN₄O₃Si requires: 448. found: 449 (M+H⁺). Rₜ=1.7 min (UPLC: column Acquity BEH C₁₈ 1.7 mm 2.1*50 mm; gradient: 10% to 100% AcCN in 2 min).

### Step 5: 7-(4-azido-2-deoxy-2-fluoro-β-D-arabinofuranosyl)-7H-pyrrolo[2,3-d]pyrimidin-4-amine

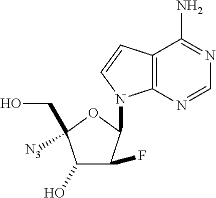

A freshly prepared solution of DMDO in acetone (0.07M, 3 eq) was added drop wise to a 0.03 M solution of 7-{3-O-[tert-butyl(dimethyl)silyl]-2,5-dideoxy-2-fluoro-β-D-threo-pent-4-enofuranosyl}-N-(2,2-dimethylpropanoyl)-7H-pyrrolo[2,3-d]pyrimidin-4-amine from Step 4 in acetone at −30° C. The mixture was stirred at the same temperature for 1 h. The volatiles were then removed under reduced pressure and the crude epoxide was dissolved in dry DCM (0.03 M) and cooled to −78° C. SnCl₄ (3 eq) and TMS-N₃ (3 eq) were added and the resulting reaction mixture was stirred 1 hour at −78° C. and 30 min to RT. The reaction was quenched at 0° C. with a few drops of 2N solution of NH₃ in MeOH 2N until pH=7. The brown slurry was filtered over a short pad of Celite and the filtrate concentrated under reduced pressure. The crude epimeric mixture of 7-{4-azido-3-O-[tert-butyl(dimethyl)silyl]-2-deoxy-2-fluoro-β-D-threo-pentofuranosyl}-N-(2,2-dimethylpropanoyl)-7H-pyrrolo[2,3-d]pyrimidin-4-amine was dissolved in dry THF and a 1N solution of TBAF in THF (4 eq) was added. The resulting mixture was stirred 2h RT, the volatiles were removed under reduced pressure and the residue was dissolved in Ammonia/Methanol 7N (0.1 M) and stirred for 48 hours at 40° C. in a pressure sealed tube. The volatiles were then removed under reduced pressure and the crude material co-evaporated twice with water. The title compound was isolated after preparative RP-HPLC purification as the more polar epimer (9% over 4 steps) (column: Atlantis T3 OBD 190×150 mm/5 μM; mobile phase: H₂O/MeCN/0.1% TFA; gradient: 2 min 5% MeCN, 14 min 5% to 50%). ¹H-NMR (400 MHz, CD₃CN/D₂O) δ 8.02 (s, 1H), 7.32 (bs, 1H), 6.68 (d, J 3.6, 1H), 6.61 (dd, J₁ 21.6, J₂ 3.6, 1H), 5.16 (app dt, J1 53.6, J2=6.0, 1H), 4.52 (dd, J₁ 21.2, J₂6.0, 1H), 3.65 (s, 2H); ¹⁹F-NMR (400 MHz, CD₃CN/D₂O) δ−204.73; MS (ES⁺) C₁₄H₁₅FN₆O₄ requires: 309. found: 310 (M+H⁺).

### EXAMPLE 2

### (Table 1, entry 2): 7-(4-azido-2-deoxy-2-fluoro-β-D-arabinofuranosyl)-5-fluoro-7H-pyrrolo[2,3-d]pyrimidin-4-amine

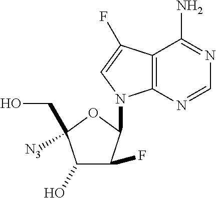

The title compound can be obtained in a similar manner to that described for Example 1, according to the synthetic routes described in Scheme 1 and Scheme 2.

NMR (400 MHz, CD₃CN/D₂O) δ 8.13 (s, 1H), 7.26 (bs, 1H), 6.77 (m, 1H), 5.27 (app dt, J₁ 53.7, J₂ 6.1, 1H), 4.62 (dd, J₁ 21.0, J₂6.3, 1H), 3.76 (s, 2H); ¹⁹F-NMR (400 MHz, CD₃CN/D₂O) δ−204.5 (1H), −166.5 (1H); MS (ES⁺) C₁₁H₁₁F₂N₇O₃ requires: 327. found: 328 (M+H).

### EXAMPLE 3

### (Table 1, entry 3): 9-(4-azido-2-deoxy-2-fluoro-b-D-arabinofuranosyl)-9H-purin-6-amine

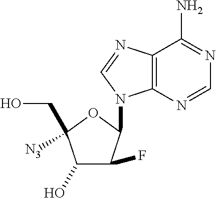

The title compound can be obtained in a similar manner to that described for Example 1, according to the synthetic routes described in Scheme 1 and Scheme 2.

NMR (400 MHz, CD₃CN/D₂O) δ 8.35 (s, 1H), 6.71 (m, 1H), 5.28 (app dt, J₁ 53.2, J₂ 6.0, 1H), 4.61 (dd, J₁ 20.8, J₂ 6.4, 1H), 3.90 (s, 2H); ¹⁹F-NMR (400 MHz, CD₃CN/D₂O) δ−202.9 (1H), −166.5 (1H); MS (ES⁺C₁₀H₁₁FN₈O₃ requires: 310. found: 311 (M+H⁺).

TABLE 1

| Name, Example | | Exact | Observed |
|---|---|---|---|
| and Scheme # | Structure | Mass | Mass |
| 7-(4-azido-2- deoxy-2- fluoro-β-D- arabino- furanosyl)- 7H-pyrrolo [2,3-d] pyrimidin-4- amine Example 1, Scheme 1 | 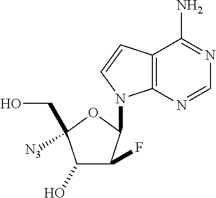 | 309 | 310 |
| 7-(4-azido-2- deoxy-2- fluoro-β-D- arabino- furanosyl)- 5-fluoro-7H- pyrrolo[2,3-d] pyrimidin-4- amine Example 2, Scheme 2 | 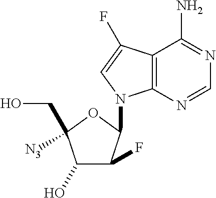 | 327 | 328 |
| 9-(4-azido- 2-deoxy- 2-fluoro-b- D-arabino- furanosyl)- 9H-purin- 6-amine Example 3, Scheme 3 | 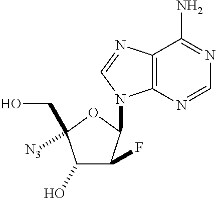 | 310 | 311 |

The compounds of the present invention were also evaluated for cellular toxicity and anti-viral specificity in the counterscreens described below.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for severity of the HCV infection. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

### Biological Assays

The assays employed to measure the inhibition of HCV NS5B polymerase and HCV replication are described below.

The effectiveness of the compounds of the present invention as inhibitors of HCV NS5B RNA-dependent RNA polymerase (RdRp) was measured in the following assay.

### A. Assay for Inhibition of HCV NS5B Polymerase:

This assay was used to measure the ability of the nucleoside derivatives of the present invention to inhibit the enzymatic activity of the RNA-dependent RNA polymerase (NS5B) of the hepatitis C virus (HCV) on a heteromeric RNA template.

### Procedure:

Assay Buffer Conditions: (52.5 μL—total/reaction)

20 mM Tris, pH 7.5

45 mM KCl

2 mM MgCl₂

0.01% Triton X-100

1 μg BSA, DNase Free

1 mM DTT

2 nM DC55-1b.BK or 10 nM DC55-2b.2

20 nM heterogeneous template dCoh

UTP 1 uM

ATP 1 uM

CTP 1 uM

GTP 1 uM

3H-UTP 1,000,000 cpm

2.5 μl/reaction inhibitor compound in H₂O

The compounds were tested at various concentrations up to 100 μM final concentration. Nucleoside derivatives were pipetted into wells of a 96-well plate. The enzyme diluted in the reaction buffer was pipetted into the wells and incubated at room temperature for 10 minutes; then the template dCoh was added and incubated for 10 minutes at room temperature. The reaction was initiated by addition of a mixture of nucleotide triphosphates (NTP's), including the radiolabeled UTP, and allowed to proceed at room temperature for 2 hours. Blank samples were done omitting the dCoh template. The reaction was quenched by addition of 50 ul TCA 20% (trichloroacetic acid)/NaPPi 20 mM and the plates were put in ice for 5 minutes. Then, the mixtures were filtered onto Unifilter GF/B 96-well plates (PerkinElmer), washed with TCA 2.5%. 50 ul/well of scintillator solution (Microscint 20, PerkinElmer) were added and the plates were counted in a scintillator counter.

The percentage of inhibition was calculated according to the following equation:

% Inhibition=[1−(cpm in test reaction−cpm in blank)/(cpm in control reaction−cpm in blank)]×100.

Representative compounds were tested in the HCV NS5B polymerase assay.

### Activity Ranges:

### +++: <1 μM; ++: <50 μM; +: >50 μM;

### B. Assay for Inhibition of HCV RNA Replication:

The compounds of the present invention were also evaluated for their ability to affect the replication of Hepatitis C Virus RNA in cultured hepatoma (HuH-7) cells containing a subgenomic HCV Replicon. The details of the assay are described below. This Replicon assay is a modification of that described in V. Lohmann, F. Korner, J-O. Koch, U. Herian, L. Theilmann, and R. Bartenschlager, “Replication of a Sub-genomic Hepatitis C Virus RNAs in a Hepatoma Cell Line,” Science 285:110 (1999).

### Protocol:

The assay was an in situ Ribonuclease protection, Scintillation Proximity based-plate assay (SPA). 10,000-40,000 cells were plated in 100-200 μL of media containing 0.8 mg/mL G418 in 96-well cytostar plates (Amersham). Compounds were added to cells at various concentrations up to 100 μM in 1% DMSO at time 0 to 18 h and then cultured for 24-96 h. Cells were fixed (20 min, 10% formalin), permeabilized (20 min, 0.25% Triton X-100/PBS) and hybridized (overnight, 50° C.) with a single-stranded ³³P RNA probe complementary to the (+) strand NS5B (or other genes) contained in the RNA viral genome. Cells were washed, treated with RNAse, washed, heated to 65° C. and counted in a Top-Count Inhibition of replication was read as a decrease in counts per minute (cpm).

Human HuH-7 hepatoma cells, which were selected to contain a subgenomic replicon, carry a cytoplasmic RNA consisting of an HCV 5′ non-translated region (NTR), a neomycin selectable marker, an EMCV IRES (internal ribosome entry site), and HCV non-structural proteins NS3 through NS5B, followed by the 3′ NTR.

Representative compounds were tested in the HCV replication assay and results are reported as EC50 activity ranges in Table 2 below:

TABLE 2

| | | EC₅₀ | CC₅₀ |
|---|---|---|---|
| # | Name | (μM) | (μM) |
| 1 | 7-(4-azido-2-deoxy-2-fluoro-β-D-arabinofuranosyl)- | +++ | >100 |
| | 7H-pyrrolo[2,3-d]pyrimidin-4-amine | | |
| 2 | 7-(4-azido-2-deoxy-2-fluoro-β-D-arabinofuranosyl)-5- | + | >100 |
| | fluoro-7H-pyrrolo[2,3-d]pyrimidin-4-amine | | |
| 3 | 9-(4-azido-2-deoxy-2-fluoro-b-D-arabinofuranosyl)- | − | >100 |
| | 9H-purin-6-amine | | |

Activity ranges:
+++: <20 μM;
+: 20 < EC₅₀ < 100 μM;
−: >100 μM;

### Example of a Pharmaceutical Formulation

As a specific embodiment of an oral composition of a compound of the present invention, 50 mg of any one of the Examples is formulated with sufficient finely divided lactose to provide a total amount of 580 to 590 mg to fill a size 0 hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for severity of the HCV infection. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A method for the treatment of HCV infection in a mammal in need of such treatment, said method comprising administering to said mammal an effective amount of a compound of formula (I): 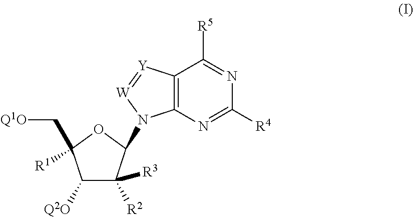
and pharmaceutically acceptable salts thereof; wherein:
Y is a group CR⁶ wherein (i) R⁶ is hydrogen, CHO, nitrile, ethynyl, a group CONH₂ optionally substituted by one or two C₁₋₃ aliphatic groups, or a C₁₋₃ aliphatic group optionally substituted by fluoro or (ii) R⁶ is amino optionally substituted by COR⁷, wherein R⁷ is a C₁₋₆ aliphatic group or phenyl; or Y and R⁵ join to form a tricyclic ring having the formula: 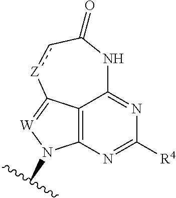
wherein the dotted line represents a single or double bond and Z is CH when the dotted line represents a double bond or Z is O or CH, when the dotted line represents a single bond;
W is N or CH;
R¹ is azido, ethynyl, nitrile or a C₁₋₃ aliphatic group optionally substituted by fluoro;
R² is hydrogen or fluoro;
R³ is hydrogen, fluoro, hydroxyl, a C₁₋₃ alkoxy group or a C₁₋₃ aliphatic group, wherein said C₁₋₃ aliphatic group is optionally substituted by fluoro;
R⁴ is hydrogen, amino or hydroxyl;
R⁵ is hydroxyl or amino;
Q¹ is hydrogen, monophosphate, diphosphate, triphosphate or a group Q³;
Q² is hydrogen or a group Q⁴;
Q³ is any primary hydroxy protecting group; and
Q⁴ is any secondary hydroxy protecting group

2. The method of claim 1, wherein the compound of formula (I) is: 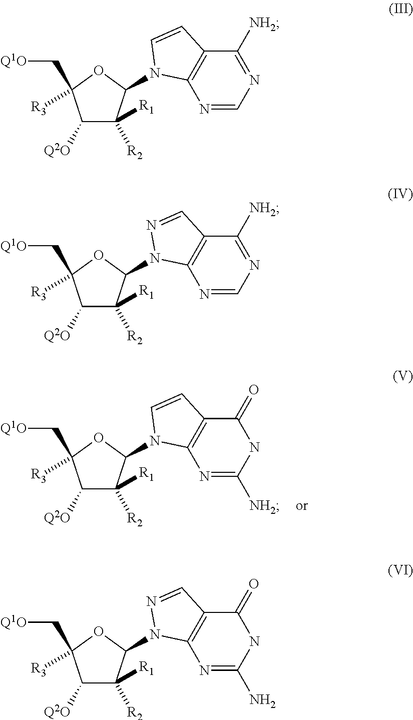
and pharmaceutically acceptable salts thereof.

3. The method of claim 1 wherein for the compounds of formula (I). Q¹ is Q³, and Q³ is selected from C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl and a monophosphate prodrug residue having the formula: 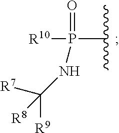
R⁷ is hydrogen or a C₁₋₆alkyl group that can be optionally substituted with one substituent selected from fluoro, hydroxy, methoxy, amino, carboxy, carbamoyl, guanidino, mercapto, methylthio, 1H-imidazolyl, and 1H-indol-3-yl; or R⁷ is phenyl, benzyl or phenethyl, wherein said phenyl, benzyl and phenethyl groups can each be optionally substituted with one to two substituents, each independently selected from halogen, hydroxy, and methoxy;
R⁸ is hydrogen or methyl, or R⁷ and R⁸ together with the carbon atom to which they attached, join to form a 3- to 6-membered aliphatic spirocyclic ring system;
R⁹ is aryl, arylalkyl, heteroaryl, 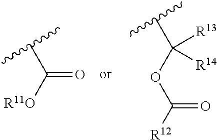
wherein R¹¹ is C₁₋₁₆alkyl, C₂₋₂₀alkenyl, —(CH₂)₀₋₄C₇₋₉-cycloalkyl, —(CH₂)₀₋₄C₃₋₉cycloalkenyl or adamantyl, any of which can each be optionally substituted with one to three substituents, each independently selected from halogen, hydroxy, carboxy, C₁₋₄alkoxy, trifluoromethyl and —(CH₂)₀₋₄NR¹⁵R¹⁶ wherein (i) R¹⁵ and R¹⁶ are independently selected from hydrogen and C₁₋₆alkyl; or (ii) any R¹⁵ and R¹⁶ groups that are attached to the same nitrogen atom, together with the common nitrogen atom to which they are attached, join to form a 4- to 7-membered heterocyclic ring containing up to 2 heteroatoms independently selected from N, O and S, wherein said 4- to 7-membered heterocyclic ring is optionally substituted by C₁₋₆ alkyl;
R¹⁰ is hydroxy or a group OR¹⁶ wherein R¹⁶ is —C₂OC(O)R¹⁷ or —CH₂CH₂SR¹⁷ wherein R¹⁷ is C₁₋₆ alkylcarbonyl optionally substituted by a hydroxyl group, or R¹⁶ is —(CH₂)₂₋₄—O—(C₂)₁₋₁₇CH₃, phenyl, naphthyl, pyridinyl, pyrimidinyl, pyrazinyl, pyridazinyl, indolyl, quinolinyl, or isoquinolinyl wherein said phenyl, naphthyl, pyridinyl, pyrimidinyl, pyrazinyl, pyridazinyl, indolyl, quinolinyl, or isoquinolinyl is optionally substituted with one to five substituents, each independently selected from halogen, C₁₋₄ alkyl, C₁₋₄ alkoxy, C₁₋₄ alkylthio, cyano, nitro, amino, carboxy.
trifluoromethyl, trifluoromethoxy, C₁₋₄ alkylamino, di(C₁₋₄ alkyl)amino, C₁₋₄ alkylcarbonyl, C₁₋₄ alkylcarbonyloxy, and C₁₋₄ alkyloxycarbonyl; or Q² is Q⁴, and R¹⁰ and Q⁴ join to form a cyclic phosphate group;
R¹² is C₆₋₁₆alkyl, C₂₋₂₀alkenyl, (CH₂)₀₋₂C₇₋₉cycloalkyl, (CH₂)₀₋₂C₃₋₉cycloalkenyl, OC₁₋₆alkyl or adamantyl; and
R¹³ and R¹⁴ are each independently selected from hydrogen and C₁₋₆alkyl;
or R¹³ and R¹⁴ together with the carbon atom to which they attached, join to form a 3- to 6-membered aliphatic spirocyclic ring system;
and/or Q⁴ is methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl and an amino acyl residue having the formula: 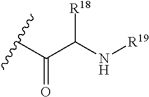
wherein R¹⁸ is hydrogen, C₁₋₅ alkyl or phenylC₀₋₂ alkyl; and R¹⁹ is hydrogen. C₁₋₄ alkyl, C₁₋₄ alkylsulfonyl or phenylC₀₋₂ alkylsulfonyl, or a group COR²⁰ wherein R²⁰ is C₁₋₄ alkyl optionally substituted by phenyl, C₁₋₄ alkoxy optionally substituted by phenyl, C₁₋₄alkylamino optionally substituted by C₁₋₄ alkyl optionally substituted by phenyl.

4. The method of claim 1 wherein for the compounds of formula (I), Q¹ is selected from hydrogen, monophosphate, diphosphate, a triphosphate, C₁-C₁₆-alkylcarbonyl or a monophosphate prodrug residue having the formula: 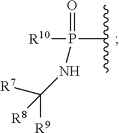
wherein R⁷ is hydrogen, methyl or benzyl;
R⁸ is hydrogen or methyl;
R⁹ is phenyl, CO₂R¹¹ or CR¹³R¹⁴OC(O)R¹²;
R¹⁰ is hydroxyl or OR¹⁶;
R¹⁶ is an aromatic or heteroaromatic ring or CH₂CH₂SR¹⁷; and
R¹⁷ is C₁-C₆ alkylcarbonyl, optionally substituted with a hydroxyl group.

5. The method of claim 1, wherein for the compounds of formula (I). Q² is selected from hydrogen, C₁-C₁₆-alkylcarbonyl and an amino acyl residue having the formula: 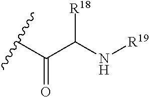
wherein R¹⁸ is hydrogen or C₁-C₅ alkyl, and R¹⁹ is hydrogen.

6. The method of claim 1, wherein for the compounds of formula (I), R¹ is azido or ethynyl.

7. The method of claim 1, wherein for the compounds of formula (I). R² is hydrogen and R³ is fluoro.

8. The method of claim 1, wherein for the compounds of formula (I), R⁴ is hydrogen and R⁵ is amino.

9. The method of claim 1, wherein for the compounds of formula (I), Y is CH and W is CH.

10. A compound of the formula (VII): 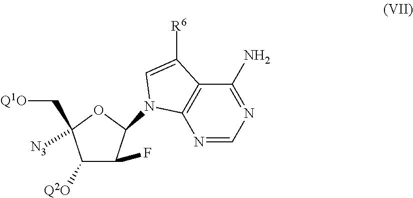
and pharmaceutically acceptable salts thereof, wherein R⁶, Q¹ and Q² are as defined in claim 1.

11. A compound according to claim 10 wherein R⁶ is hydrogen.

12. (canceled)

13. The compound having the structure: 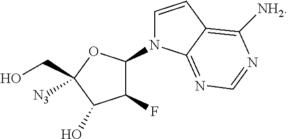
and pharmaceutically acceptable salts thereof.

14. (canceled)

15. (canceled)

16. A method for inhibiting RNA-dependent RNA viral polymerases, a method for inhibiting RNA-dependent RNA viral replication, and/or a method for treating RNA-dependent RNA viral infections in a mammal in need thereof comprising administering to the mammal a therapeutically effective amount of a compound of claim 1.

17. A pharmaceutical formulation comprising a compound of claim 10 and a pharmaceutically acceptable carrier.
